(19) United States
(12) Patent Application Publication
(10) Pub. No.: US 2017/0326123 A1
(43) Pub. Date: Nov. 16, 2017

(54) Throat solution for treatment of cold, flu and sore throat

(71) Applicant: Laurence Martin Shanley, Miami, FL (US)

(72) Inventor: Laurence Martin Shanley, Miami, FL (US)

(21) Appl. No.: 15/153,678

(22) Filed: May 12, 2016

(51) Int. Cl. A61K 31/4425; A61K 36/889; A61K 36/87; A61K 36/736; A61K 36/63; A61K 36/54; A61K 9/00; A61K 36/48; A61K 36/31; A61K 36/286; A61K 36/28; A61K 31/045; A61K 9/08; A61K 36/534; A61K 36/899

(52) U.S. Cl. CPC A61K 31/4425; A61K 36/899; A61K 36/889; A61K 36/87; A61K 36/736; A61K 36/63; A61K 36/54; A61K 9/006; A61K 36/48; A61K 36/31; A61K 36/286; A61K 36/28; A61K 31/045; A61K 9/08; A61K 36/534

(57) ABSTRACT

A composition of isopropyl or ethyl alcohol, Cetylpyridinium Chloride and essential oils that enable the composition to have a prolonged antimicrobial effect by reducing the normal rate of alcohol dehydration, reducing toxicity and increasing duration of efficacy with proper application to oral cavity, oropharynx and directly pharyngeal tonsils.

### DESCRIPTION OF INVENTION

A composition of isopropyl or ethyl alcohol, Cetylpyridinium Chloride and essential oils that enable the composition to have a prolonged antimicrobial effect by reducing the normal rate of alcohol dehydration, reducing toxicity and increasing duration of efficacy with proper application to oral cavity, oropharynx and directly to pharyngeal tonsils. Bacterial and viral invitro time kill studies have shown that by diluting alcohol with oil actually increases the efficacy and reduces toxicity. Shelf alcohol whether it is 91%, 70% or 50% have all shown to evaporate too fast inhibiting the chemical activity to destroy the virus or bacteria. This teaching shows that diluting alcohol with a specific oil base, even hard to kill non-enveloped virus (Rhinovirus, Common Cold) are destroyed.

A solution comprising: an isopropyl alcohol or ethyl alcohol component between (50-70% by weight and 15-35% by total volume cetylpyridinium chloride (cpc) not exceeding 0.10% by volume; a sesame oil or alternative oil such as sunflower oil, safflower oil, peanut oil, olive oil, coconut oil, soybean oil, avocado oil, corn oil, almond oil, canola oil, grape oil component not exceeding 80% by volume; a lemon oil or lemon balm component not exceeding 4% by volume; a peppermint oil or alternative wintergreen oil, mint oil, spearmint oil, eucalyptus oil not exceeding 6%; aloe vera oil extract not exceeding 5%), for supplementing and neutralizing the dehydrating effect, mucosal irritation and toxicity of said alcohol; a proprietary natural and artificial strawberry flavor additive or alternative, cherry, grape, lemon, lime, orange, bubble gum, berry berry, coffee, vanilla, chocolate, mint, blueberry and does not exceed 8%. Also a proprietary emulsification process can be utilized to create a soluble final product as an alternative to manual mixing before application. A proprietary high output flow control for the spray tip is utilized for necessary therapeutic administration to the oropharynx and pharyngeal tonsils. The solution may also be applied with a swab type device directly to the pharyngeal tonsils using a moderate rotational spread. As described here the teaching shows that it is imperative to reduce the dehydrating effect of the alcohol with the essential oils and the solution must be applied correctly with appropriate friction and or direct jet stream.

### FIELD OF THE INVENTION

The present invention lies in the pharmacological field of mucosal disinfectants. More specifically, the invention pertains to a non-toxic mucosal disinfectant that is effective against various pathogenic organisms that cause infectious processes in the throat.

Sore throats are common. Most of the time the soreness is worse in the morning and improves as the day progresses. Like colds, the vast majority of sore throats are caused by viral infections, such as a cold or flu. This means most sore throats will not respond to antibiotics. Many people have a mild sore throat at the beginning of every cold. When the nose or sinuses become infected, drainage can run down the back of the throat and irritate it, especially at night. Or, the throat itself can be infected.

Some viruses can cause specific types of sore throat. For example, Coxsackievirus sometimes causes blisters in the throat, especially in the late summer and early fall. Mononucleosis and the flu can also cause specific viral throat infections.

Strep throat is the most common bacterial cause of sore throat. Because strep throat can occasionally lead to rheumatic fever, antibiotics are given. Strep throat often includes a fever (greater than 101° F.), white, draining patches on the throat, and swollen or tender lymph glands in the neck. Children may have a headache and stomach pain.

A sore throat is less likely to be strep throat if it is a minor part of a typical cold (with runny nose, stuffy ears, cough, and similar symptoms). Strep cannot be accurately diagnosed by looking at the throat alone. It requires a laboratory test.

Sometimes breathing through the mouth will cause a sore throat in the absence of any infection. During the months of dry winter air, some people will wake up with a sore throat most mornings. This usually disappears after having something to drink. A sore throat may also be caused by something stuck in the throat or allergies (allergic rhinitis).

With a sore throat, sometimes the tonsils or surrounding parts of the throat are inflamed. Either way, removing the tonsils to try to prevent future sore throats is not recommended for most children.

### SUMMARY OF THE INVENTION

The present invention relates to a throat (mucosal) disinfectant topically applied or by spray that effectively destroys various pathogenic organisms that cause sore throat. The solution coats the throat and specifically the pharyngeal tonsil region and does not rapidly evaporate thus maintaining antimicrobial efficacy for a duration long enough to reduce or even eliminate active infection and the symptoms associated.

Time Kill Studies

| | |
|---|---|
| Rhinovirus (Common Cold) | 98.33% |
| Coronavirus (SARS) | 99.99% |
| Influenza A (Bird Flu) | 99.99% |
| Adenovirus | 99.99% |
| Staphylococcus Aureus | 99.99% |
| Streptococcus Pneumoniae | 99.99% |
| Streptococcus Pyogenes | 99.99% |
| Hemophilius Influenzae | 99.99% |

SKIN IRRITATION STUDY: Under the conditions of the study, irritation was observed on the skin of the rabbits. The primary irritation index was calculated to be 1.08. The test article would not be considered a primary irritant to the skin since the empirical score was less than 5.00. It has been determined by the FDA that score results below 5.00 on rabbits is very safe for human skin.

DERMAL TOXICITY STUDY: Under the conditions of the study, there was no mortality or significant evidence of toxicity observed in the rabbits. The test article would not be considered toxic at a dose of 2 g/kg by the dermal route in the rabbits.

### Comparison To Common use Mouthwash Products:

Due to the extensive background in research and development of Dr. Baugh, Custom Biologicals is the industry leader in selecting and adapting natural microorganisms for a multitude of industrial, agricultural, and domestic applications. Additionally, CBI has the strength of the unique proprietary production methods developed by Dr. Baugh for bacterial products.

The antimicrobial solution did not inactivate the bacillus cereus spore, however it was very effective for killing the vegetative cells. There are many mouthwash type products that will kill vegetative cells, but not at the speed of your solution.

We claim:

1. The antimicrobial composition of mixed component for topical application in the throat, comprising: an ethyl or isopropyl alcohol component between 15%-35% by weight and 50%-70% by volume; a composition of various base and essential oils between 60%-80% by weight; Cetylprydnium Chloride no greater than 10% by weight; a proprietary flavor additive no greater than 5% by weight for supplementing, neutralizing, activating and reducing the dehydrating effect of said alcohol thus enabling the necessary contact time effectively kill the germs that cause sore throat without mucosal toxicity or irritation.

2. The peppermint, spearmint, menthol or like essential oil no greater than 4% by weight, in combination with solution, neutralizes the undesirable affect from alcohol aroma, temporarily reduces associated pain from a throat infection, temporarily improves sensation of breathing and provides temporary flavor enhancement.

3. The composition is created with a high viscosity and is applied with direct high velocity or friction in order to penetrate affected or targeted tissues resulting in a greater antimicrobial effect.

4. The said composition according to claim 1, wherein said Cetylprydnium Chloride can be added to increase the antimicrobial effect.

5. The said composition according to claim 1, wherein said essential oils are added for increasing natural antimicrobial effect.

6. The said composition according to claim 3, a proprietary emulsification process may be utilized as an alternative to manual mixing.

7. The said composition according to claim 1, wherein said base oils between 60% and 80% by weight such as sunflower oil, safflower oil, peanut oil, olive oil, coconut oil, soybean oil, avocado oil, corn oil, almond oil, canola oil, grape oil component can be utilized individually or in a combination and have shown to have natural antiseptic, absorption and tissue regeneration properties.

8. The said composition according to claim 1, wherein said proprietary variant viscosity flavor additives such as strawberry or alternative, cherry, grape, lemon, lime, orange, bubble gum, berry berry, coffee, vanilla, chocolate, mint, blueberry not exceeding 8% by weight is utilized, which enhances or aids in neutralizing the alcohol affect or aroma.

9. The said composition according to claim 1, wherein said essential lemon oil or alternate lemon balm no greater than 4% by volume, may be added to enhance antimicrobial efficacy, deliver nutrients to tissues and helps to neutralize the after taste of alcohol and oils.

10. The said composition according to claim 1, wherein said essential aloe vera oil extract no greater than 4% by volume, is added to enhance tissue healing and regeneration.

11. The said composition according to claim 1, wherein the said antimicrobial solution has shown to reduce or eliminate the infection in the throat better than other solutions including, mouthwash, saltwater solution, over the counter throat sprays and peroxide base products.

12. The said composition according to claim 1, wherein the said antimicrobial solution has shown to reduce or eliminate germs that cause bad breathe, the oil base helps reduce dehydration of active ingredients on the tongue and pharyngeal mucosa.

13. The said composition according to claim 1, wherein the said composition has shown to kill common respiratory bacteria in time kill studies (99.9% effective). Alcohol products for the throat have never presented this activity because of rapid evaporation or dilution with saliva.

14. The said composition according to claim 1, wherein the said composition has shown to kill respiratory virus in time kill studies 98.33-99.9%. Alcohol products for the throat have never demonstrated this type of efficacy for virus because of rapid evaporation or dilution.

15. The said composition according to claim 1, wherein the said composition even in the highest dosage formulary has shown to not produce and dermal irritation or dermal toxicity to mucosal tissues. The alcohol is diluted by up to thirty percent with water and then that mixture is diluted by up to 85% with oil. No adverse side effects have ever been reported during clinical testing.
